(19) United States
(12) Patent Application Publication
(10) Pub. No.: US 2025/0082699 A1
(43) Pub. Date: Mar. 13, 2025

(54) Bifidobacterium Longum Subsp. Infantis CCFM1269 and Application thereof

(71) Applicant: Jiangnan University, Wuxi (CN)

(72) Inventors: Bo YANG, Wuxi (CN); Wei CHEN, Wuxi (CN); Mengfan DING, Wuxi (CN); Bowen LI, Wuxi (CN); Haiqin CHEN, Wuxi (CN); Jianxin ZHAO, Wuxi (CN); Hao ZHANG, Wuxi (CN)

(21) Appl. No.: 18/959,811

(22) Filed: Nov. 26, 2024

Related U.S. Application Data
(63) Continuation of application No. PCT/CN2023/126796, filed on Oct. 26, 2023.

(30) Foreign Application Priority Data
Nov. 22, 2022 (CN) 2022114738059
Jul. 27, 2023 (CN) 2023109325648

(51) Int. Cl. A61K 35/745; A61K 35/00; C12N 1/20

(52) U.S. Cl. CPC A61K 35/745; C12N 1/20; A61K 2035/115

(57) ABSTRACT

The disclosure provides Bifidobacterium longum subsp. infantis CCFM1269 and an application thereof, and belongs to the technical field of microorganisms. The B. longum subsp. infantis CCFM1269 provided by the disclosure is a food safety strain, which has the effects of regulating the Th1/Th2 balance and the IgA content of a mammal in an early life stage, enhancing the skeletal development, improving the femur length and skeletal steady state in the early life stage and the like, and thus the strain has a huge application prospect. Only a culture medium and control of some culture conditions are needed in a culture process of the strain, so that the cost is relatively low, and industrial production is easily implemented.

### TECHNICAL FIELD

The disclosure relates to B. longum subsp. infantis CCFM1269 and an application thereof, and belongs to the technical field of microorganisms.

### BACKGROUND

B. longum subsp. infantis is a main Bifidobacterium colonist in an infantile intestinal tract, exhibiting advantages in prompting early life health. With the rise of the incidence of various non-infectious diseases, people have developed a keen interest in discovering the causes of diseases and paid attention to the early immune establishment to reduce the disease incidence risk in a later stage. Particularly in an infantile stage with strong plasticity, a reasonable nutrition supply may reduce the occurrence of diseases in the growth process to the maximum extent. After birth, thanks to factors such as hormones and progesterone, infants show a preference for an immune type 2 of a T ancillary cell, which is the primary cause of an increased risk of allergy, asthma, and other diseases in childhood. Besides, an imbalance of intestinal flora of infants is very common in modern society, which may be a factor causing an increase in the incidence of immune-mediated diseases. Breast-fed infants may resist diarrhea, allergy, asthma, and inflammatory bowel disease. Breast milk not only provides infants with essential nutrition, but also prompts establishment of the intestinal flora. Therefore, it is necessary to excavate a microbiota capable of resisting the occurrence of allergy, asthma, and other possible immune diseases and promoting the immune system.

Bone development is very important in the growth process. During childhood and adolescence, the skeleton grows and develops constantly, the bone mass, bone density, and bone quality will increase as age increases. After becoming an adult, the growth and development of the skeleton will stop gradually, and at this time, the skeleton will enter a steady state. In the growth process, good bone development can bring many benefits as follows: (1) preventing fracture: good bone development may make the skeleton more robust so that the fracture risk is reduced; (2) preventing osteoporosis: osteoporosis is a disease with a too-low bone quality, and good bone development may reduce the probability of occurrence of osteoporosis; (3) improving the athletic ability: good bone development may improve the strength and stability of the skeleton, so as to enhance the athletic ability and physical power; and preventing abnormal postures and campylorrachis scoliosa: (4) good bone development may prompt normal postures and growth and development of the spine and reduces problems of abnormal postures and campylorrachis scoliosa. Therefore, good bone development plays an important role in the growth process. Timely measures should be taken during childhood and adolescence to promote bone development and maintain bone health. After becoming an adult, a healthy diet and a reasonable mode of exercise should also be kept to maintain the health condition of the skeleton.

The metabolism of bones is balanced by the formation and absorption of bones. When bone formation predominates, It is characterized by bone formation and metabolism, and conversely, absorption and catabolismare central. It is understood that the metabolic pathway, the immune system, and the hormonal environment of a host may affect such bone metabolic balance. Probiotics have been found to play an extremely important role in maintaining the microbial balance of the intestine, prompting intestinal health, enhancing immunity. There is a close relationship between the intestinal health and the bone development. The probiotics may affect bone development through a series of mechanisms, including prompting calcium absorption, improving the intestinal flora, reducing the inflammatory reaction, and the like. Therefore, it is quite significant to research the influences of probiotics on bone development. The current research found that Lactobacillus rhamnosus, Lactobacillus paracasei, Lactobacillus reuteri, Lactobacillus plantarum, and Lactobacillus acidophilus have certain regulating effects on skeletal and muscular parameters such as reducing bone absorption, increasing IGF-1 in serum, increasing Treg cells, and reducing the quantity of Th17 cells. Present research on Bifidobacterium mainly focuses on the improving effect on bone loss. For example, Bifidobacterium pseudocatenulatum CECT7765 improves the bone mass loss caused by obesity, the B. longum increases the bone density by regulating Bmp-2, and there have not been many reports on how the B. longum subsp. infantis, as a dominant strain for the infantile intestine, affects bone development. Therefore, it is of great importance to find the B. longum subsp. infantis with the effect of regulating skeletal development.

### SUMMARY

The disclosure provides B. longum subsp. infantis, which is B. longum subsp. infantis CCFM1269, taxonomically named as B. longum subsp. infantis. The B. longum subsp. infantis has been preserved in Guangdong Microbial Culture Collection Center (GDMCC) on Sep. 26, 2022, with a preservation number of GDMCC No: 62839 and a preservation address: Floor 5, Building 59, Yard 100, Middle Xianlie Road, Guangzhou.

The disclosure further provides a composition including the B. longum subsp. infantis CCFM1269; and the composition is a probiotic preparation, a leavening agent, a health care product, or a medicine.

In an embodiment, a viable count of the B. longum subsp. infantis in the probiotic preparation is not lower than 1×10⁹ CFU/mL or 1×10⁹ CFU/g.

In an embodiment, the probiotic preparation is a bacterial suspension of the B. longum subsp. infantis CCFM1269.

In an embodiment, a method for preparing the leavening agent includes: inoculating the B. longum subsp. infantis CCFM1269 to a culture medium, culturing the culture medium for 24-48 h at 37° C. to obtain a culture solution; centrifuging the culture solution to obtain thalli; and resuspending the thalli with normal saline to obtain the leavening agent.

In an embodiment, the culture medium is an MRS culture medium.

In an embodiment, the viable count of the B. longum subsp. infantis in the medicine is not lower than 1×10⁹ CFU/mL or 1×10⁹ CFU/g.

In an embodiment, the medicine contains the B. longum subsp. infantis and a pharmaceutically acceptable carrier.

In an embodiment, the medicine is used to regulate Th1/Th2 balance, prompt IgA synthesis and/or enhance skeletal development.

The disclosure further provides a method of ingesting the B. longum subsp. infantis CCFM1269 or the composition into gastrointestinal environments of individuals to enhance the immunity of the individuals, improve the bone density of the individuals in an auxiliary manner, enhance the skeletal development of the individuals, and regulate the intestinal flora of the individuals.

In an embodiment, the individuals include, but are not limited to, mammals in an early life stage.

In an embodiment, the enhancing the immunity includes regulating the Th1/Th2 balance and prompting the IgA synthesis.

In an embodiment, the regulating the Th1/Th2 balance and prompting the IgA synthesis include at least one of the following effects:

(1) improving the percentage contents of B cells, T cells, and Th cells in mesenteric lymph nodes of the mammals in the early life stage;

(2) improving the contents of IFN-γ, IgG2a, IgA, and sIgA in the colons of the mammals in the early life stage and the ratio of IgG2a/IgE;

(3) improving the content of IFN-γ in serums of the mammals in the early life stage and the ratio of IgG2a/IgE;

(4) reducing the contents of IL-4 and IgE in the colons of male mammals in the early life stage;

(5) improving the relative expression level of T-bet mRNA in the colons of the mammals

in the early life stage; and

(6) improving the relative abundance of IgA-coated Alistipes in feces of the male mammals in the early life stage.

In an embodiment, the enhancing the skeletal development refers to improving the femur length and the skeletal steady stage in the early life stage, including at least one of the following effects:

(1) improving the femur lengths of the mammals in the early life stage;

(2) improving the levels of a growth hormone in the serums of the mammals in the early life stage;

(3) improving the levels of an insulin-like growth factor 1 in the serums of the mammals in the early life stage;

(4) improving the levels of osteoprotegerin in the serums of the mammals in the early life stage;

(5) improving the levels of osteocalcin in the serums of the mammals in the early life stage;

(6) improving the levels of N-terminal propeptides of type I collagens in the serums of the mammals in the early life stage; and

(7) reducing the levels of C-terminal peptides of the type I collagens in the serums of the mammals in the early life stage.

The disclosure further provides an application of the B. longum subsp. infantis CCFM1269 in preparing health care products beneficial for enhancing immunity and improving bone density in an auxiliary manner.

The disclosure further provides an application of the B. longum subsp. infantis CCFM1269 in a probiotic product regulating the Th1/Th2 balance and IgA synthesis.

In an embodiment, the product is used to improve the levels of Th1/Th2-related cell factors and immune globulins in the colons of the mammals in the early life stage.

In an embodiment, the product is used to enhance the skeletal development of the mammals in the early life stage.

In an embodiment, the product is used to regulate the intestinal flora of the mammals in the early life stage.

The disclosure further provides an application of the B. longum subsp. infantis CCFM1269 in preparing health care products beneficial to regulating the intestinal flora.

### Beneficial Effects

1. In the disclosure, the B. longum subsp. infantis CCFM1269 is screened for its role in regulating the Th1/Th2 balance and IgA secretion of the individuals, which is specifically embodied in:

(1) improving the percentage contents of B cells, T cells, and Th cells in mesenteric lymph nodes of female and male infant mice;

(2) improving the contents of IFN-γ, IgG2a, IgA, and sIgA in colons of the female and male infant mice and the ratio of IgG2a/IgE;

(3) improving the content of IFN-γ in serums of the female and male infant mice and the ratio of IgG2a/IgE;

(4) reducing the contents of IL-4 and IgE in the colons of the male infant mice;

(5) improving the expression levels of T-bet mRNAs in the colons of the female and male infant mice; and

(6) improving the relative abundance of IgA-coated Alistipes in feces of the male infant mice.

2. The B. longum subsp. infantis CCFM1269 screened out in the disclosure is a food safety strain, may be used to prepare products with the effects of regulating the Th1/Th2balance and the IgA content, and has a huge application prospect.

3. The B. longum subsp. infantis CCFM1269 provided by the disclosure further has the effect of enhancing the skeletal development and regulating the femur length and skeletal steady state, which are specifically embodied in:

(1) improving the femur lengths of the mammals in the early life stage;

(2) improving the levels of a growth hormone in the serums of the mammals in the early life stage;

(3) improving the levels of an insulin-like growth factor 1 in the serums of the mammals in the early life stage;

(4) improving the levels of osteoprotegerin in the serums of the mammals in the early life stage;

(5) improving the levels of osteocalcin in the serums of the mammals in the early life stage;

(6) improving the levels of N-terminal propeptides of type I collagens in the serums of the mammals in the early life stage; and

(7) reducing the levels of C-terminal peptides of the type I collagens in the serums of the mammals in the early life stage.

4. Only a culture medium and control of some culture conditions are needed in a culture process of the B. longum subsp. infantis provided by the disclosure, so that the cost is relatively low, and industrial production is easily implemented.

### Preservation of a Biological Material

B. longum subsp. infantis CCFM1269, which is taxonomically named as B. longum subsp. infantis, has been preserved in Guangdong Microbial Culture Collection Center (GDMCC) on Sep. 26, 2022, with a preservation number of GDMCC No: 62839 and a preservation address: Floor 5, Building 59, Yard 100, Middle Xianlie Road, Guangzhou.

### BRIEF DESCRIPTION OF FIGURES

FIG. 1 shows variations of B cells and Th cells of mesenteric lymph nodes of female infant mice in different groups, with *: p<0.05.

FIG. 2 shows variations of IFN-γ, IgG2a, IgA, sIgA, and IgG2a/IgE in colons of the female infant mice in different groups, with *: p<0.05.

FIG. 3 shows variations of IFN-γ, and IgG2a/IgE in serums of the female infant mice in different groups, with *: p<0.05.

FIG. 4 shows variations of T-bet mRNA in the colons of the female infant mice in different groups, with *: p<0.05.

FIG. 5 shows variations of percentage contents of T cells and Th cells of mesenteric lymph nodes of male infant mice in different groups, with *: p<0.05.

FIG. 6 shows variations of IgA, IgE, and IgG2a/IgE in the colons of the male infant mice in different groups, with *: p<0.05.

FIG. 7 shows variations of IgG2a and IgG2a/IgE in the serums of the male infant mice in different groups, with *: p<0.05.

FIG. 8 shows variations of T-bet mRNA in the colons of the male infant mice in different groups, with *: p<0.05.

FIG. 9 shows variations of sIgA-coated Alistipes in feces of the male infant mice in different groups, with *: p<0.05.

FIG. 10 shows femur lengths of mammals in an early life stage in different groups, with *, compared with a Control, p<0.05.

FIG. 11 shows levels of a growth hormone in serums of the mammals in an early life stage in different groups, with *, compared with the Control, p<0.05.

FIG. 12 shows levels of insulin-like 1 growth factors in the serums of the mammals in an early life stage in different groups, with *, compared with the Control, p<0.05.

FIG. 13 shows levels of osteoprotegerin in the serums of the mammals in an early life stage in different groups, with *, compared with the Control, p<0.05.

FIG. 14 shows levels of osteocalcin in the serums of the mammals in an early life stage in different groups, with *, compared with the Control, p<0.05.

FIG. 15 shows levels of N-terminal propeptides of type I collagens in the serums of the mammals in an early life stage in different groups, with *, compared with the Control, p<0.05.

FIG. 16 shows levels of C-terminal peptides of the type I collagens in the serums of the mammals in an early life stage in different groups, with *, compared with the Control, p<0.05.

### DETAILED DESCRIPTION

The disclosure will be further described below in conjunction with specific examples and drawings.

BALB/C mice involved in the examples below are purchased from Zhejiang Vital River. B. longum subsp. infantis CCFM1269 involved in the examples below are separated and obtained in Biotechnology Center, School of Food Science and Technology, Jiangnan University.

Detection reagents involved in the examples below are as follows:

ELISA kits for IFN-γ, IgG2a, IgE, IgA, and IL-4 are purchased from Fcmacs. ELISA kits for indexes such as growth hormone, osteocalcin, and osteoprotegerin are purchased from Fcmacs.

Culture media involved in the examples below are as follows:

MRS solid culture medium: 10 g/L peptone, 10 g/L beef extract, 20 g/L glucose, 2 g/L sodium acetate, 5 g/L powdery yeast, 2 g/L diammonium hydrogen citrate, 2.6 g/L K₂PO₄.3H₂O, 0.1 g/L MgSO₄.7 H₂O, 0.05 g/L MnSO₄, 1 mL/L Tween 80, and 15 g/L agar.

MRS liquid culture medium: 10 g/L peptone, 10 g/L beef extract, 20 g/L glucose, 2 g/L sodium acetate, 5 g/L powdery yeast, 2 g/L diammonium hydrogen citrate, 2.6 g/L K₂PO₄.3H₂O, 0.1 g/L MgSO₄.7 H₂O, 0.05 g/L MnSO4, and 1 mL/L Tween 80.

B. longum subsp. infantis 15TI, the original number of which is FJSWXI5TIM1, has been disclosed in a paper Research on Functional Genomes of B. Longum Subsp. Infantis and Influence thereof on DSS Induced Colonitis.

### Example 1: Screening and Strain Identification of B. longum subsp. infantis CCFM1269

### 1. Screening

Based on a breast milk sample originating from Wuxi in Jiangsu, 0.5 mL of the sample preserved in 30% (v/v) glycerin was taken and added into a 10 mL centrifuge tube filled with 4.5 mL of normal saline in a sterile environment to obtain a 10⁻¹ diluent, and the above diluting step was repeated to obtain 10⁻², 10⁻³, 10⁻⁴, 10⁻⁵, and 10⁻⁶ diluents in sequence; 100 μl of gradient diluents with different gradients were respectively sucked and coated to an MRS solid culture medium, and the diluents were cultured at 37° C. for 72 h to obtain a diluted spread plate; typical colonies on the diluted spread plate were picked for streaking on the MRS solid culture medium respectively, and the typical colonies were cultured at 37° C. for 48 h to obtain a purified colony; and the purified colony was picked and inoculated to an MRS liquid culture medium and were cultured at 37° C. for 48 h to a strain BJSWXB6MNIM1, which was named CCFM1269.

### 2. Identification

Genomes of CCFM1269 were extracted for 16S amplification, and 16S rDNA amplification conditions were as follows: 95° C. 5 min; 35 cycles (95° C. for 30 s, 55° C. for 30 s, and 72° C. for 2 min); and 72° C. for 10 min. Amplification primers: 27F/1492R. An amplification product purification and sequence alignment process were performed according to a method recorded in the literature (Turroni F et al. Exploring the Diversity of the Bifidobacterial Population in the Human Intestinal Tract[J]. Appl Environ Microb. 2009; 75 (6): 1534-45). 16S rDNA of the CCFM1269 was amplified and sequenced (by Suzhou GENEWIZ Biotechnology Co., Ltd.), and 16S rDNA sequences of the CCFM1269 obtained by sequencing analysis were aligned in Genebank, indicating that the strain was the B. longum subsp. infantis, named B. longum subsp. infantis CCFM1269, which was preserved in Guangdong Microbial Culture Collection Center (GDMCC), with a preservation number of GDMCC No: 62839.

### Example 2: Preparation of a B. longum subsp. infantis Bacterial Suspension

The B. longum subsp. infantis CCFM1269 bacteria suspension is prepared by the following method:

The B. longum subsp. infantis bacteria solution was dipped for streaking on an MRS solid culture medium and was cultured at 37° C. for 48 h to obtain a single colony;

the single colony was picked and inoculated to an MRS liquid culture medium and was cultured at 37° C. for 24 h to obtain an activating solution; and the activating solution was inoculated to the MRS liquid culture medium according to an inoculum size of 1% (v/v) and was cultured at 37° C. for 24 h to obtain a primary seed solution;

the primary seed solution was inoculated to the MRS liquid culture solution according to the inoculum size of 1% (v/v) and was cultured at 37° C. for 24 h to obtain a secondary seed solution; and

the secondary seed solution was inoculated to the MRS liquid culture medium according to the inoculum size of 1% (v/v) and was cultured at 37° C. for 24 h to obtain a bacteria solution; 6000 g of the bacteria solution was centrifuged for 15 min, and a precipitate was collected; after the precipitate was washed twice with a normal saline buffer, 6000 g of the bacteria solution was centrifuged again for 10 min to obtain thalli; and lactic acid bacteria thalli were resuspended with normal saline till the cell concentration was 1×10⁹ CFU/mL to obtain a B. longum subsp. infantis bacteria solution.

### Example 3: An influence of B. longum subsp. infantis CCFM1269 on Lymphocyte Contents in Mesenteric Lymph Nodes of Female Infant Mice

After 8 6-week old specific pathogen-free (SPF) BALB/C female mice and 4 6-week old SPF BALB/C male mice were fed 1 week under the condition of freely eating and drinking in a 12 h-12 h day/night cycle at a feeding room temperature of 22-24° C. and humidity of 40-60%, the female and male mice were mated in the proportion of 2:1; after the female mice were pregnant, the male mice were taken out; the gestation period was 3 weeks; and 1-week old infant mice after birth were subjected to intragastric administration, and were divided into a Control (intragastric administration with normal saline) and a CCFM1269 group (intragastric administration with the B. longum subsp. infantis CCFM1269).

Experiments were conducted after 1 week of adaptive feeding of animals, and the experiments lasted for 8 weeks. Specific treatments were as follows:

The Control: 1 week after birth, the mice were subjected to intragastric administration with 200 μL of normal saline; and

the B. longum subsp. infantis CCFM1269 group: 1 week after birth, the mice were subjected to intragastric administration with 10 μL of corresponding bacterial suspension, where the intragastric administration dose was 1×10⁹ CFU/mouse/day.

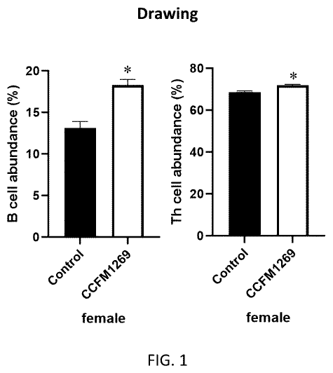

The mice were subjected to intragastric administration till they were 3-week old. After experiments, mesenteric lymph nodes of the female infant mice were taken and placed in a D-PBS to prepare a single-cell suspension. Through flow cytometry, T cells, B cells, and Th cells in the mesenteric lymph nodes were determined, and a determination result was shown in FIG. 1.

The quantities of the T cells, B cells, and Th cells in the mesenteric lymph nodes usually can reflect whether the strain can stimulate individuals to generate adaptive immunity. It can be known from FIG. 1 that the content of the B cells in the mesenteric lymph nodes of the normal group is 13.16%, and the content of the B cells in the mesenteric lymph nodes of the CCFM1269 group is 18.3%; and the percentage content of the B cells in the mesenteric lymph nodes of the female infant mice in the CCFM1269 group is significantly higher than that in the normal group (p<0.05).

The content of the Th cells in the mesenteric lymph nodes of the normal group is 68.7%, and the content of the Th cells in the mesenteric lymph nodes of the CCFM1269 group is 71.86%; and the percentage content of the Th cells in the mesenteric lymph nodes of the female infant mice in the CCFM1269 group is significantly higher than that in the normal group (p<0.05).

### Example 4: An influence of B. longum subsp. infantis CCFM1269 on Th1/Th2 Related Cell Factors and Immune Globulins of Female Infant Mice

Colons of female infant mice were taken and placed in phosphate-buffered saline and were homogenized. Homogenates of the colons of the mice in each group were determined through an ELISA kit. Th1/Th2-related cell factors and immune globulins of the colons were determined. A determination result is shown in FIG. 2.

The content of IFN-γ and the content of IgG2a are respectively primary cell factors and immune globulins of the Th1 immune type. The content of IgA and the content of sIgA are primarily mucosally-immune immune globulins. It can be known from FIG. 2 that the content of IFN-γ in the colons of the female infant mice in the CCFM1269 group is significantly higher than that in the normal group (p<0.05), the concentration of IFN-γ in the homogenates of the colons in the normal group is 2.60 ng/mg protein and the concentration of IFN-γ in the homogenates of the colons in the CCFM1269 group is 3.56 ng/mg protein.

It can be known from FIG. 2 that the content of IgG2a in the colons of the female infant mice in the CCFM1269 group is significantly higher than that in the normal group (p<0.05), the concentration of IgG2a in the homogenates of the colons in the normal group is 1.89 μg/mg protein, and the concentration of IgG2a in the homogenates of the colons in the CCFM1269 group is 2.70 μg/mg protein.

The content of IgA in the colons of the female infant mice in the CCFM1269 group is significantly higher than that in the normal group (p<0.05), the concentration of IgA in the homogenates of the colons in the normal group is 4.29 μg/mg protein, and the concentration of IgA in the homogenates of the colons in the CCFM1269 group is 7.66 μg/mg protein.

The content of sIgA in the colons of the female infant mice in the CCFM1269 group is significantly higher than that in the normal group (p<0.05), the concentration of sIgA in the homogenates of the colons in the normal group is 0.43 μg/mg protein, and the concentration of sIgA in the homogenates of the colons in the CCFM1269 is 0.84 μg/mg protein.

The increase in the ratio of IgG2a/IgE indicates that the immune type of the individuals is in transition from Th2 type to Th1 type and achieves balance gradually. IgG2a/IgE in the colons of the female infant mice in the CCFM1269 group is significantly higher than that in the normal group (p<0.05), the ratio of IgG2a to IgE in the homogenates of the colons in the normal group is 0.89 μg/mg protein, and the ratio of IgG2a to IgE in the homogenates of the colons in the CCFM1269 group is 1.42 μg/mg protein.

### Example 5: An influence of B. longum subsp. infantis CCFM1269 on Th1/Th2 Related Cell Factors and Immune Globulins in Serums of Female Infant Mice

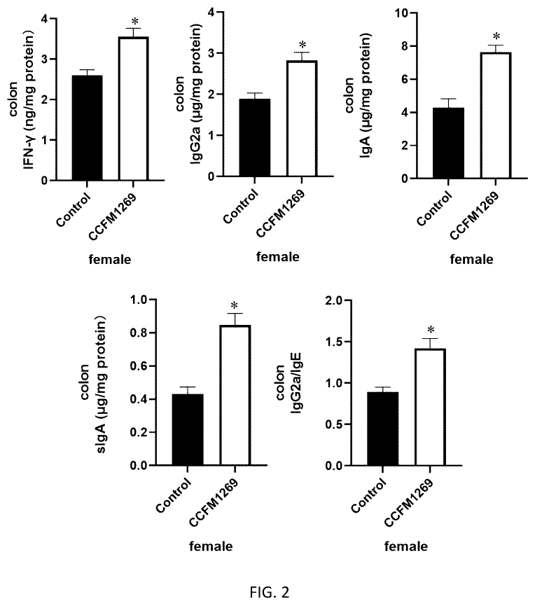

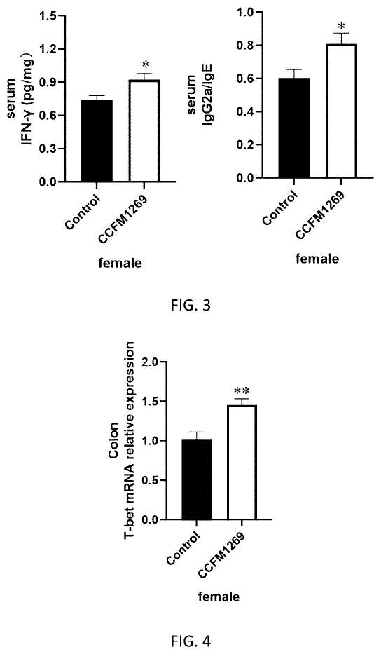

Grouping and treatment of the mice are as same as those in example 3. After experiments, blood was collected and the mice were killed. Contents of Th1/Th2-related cell factors and immune globulins in the serums of the mice in each group were determined through an ELISA kit. A detection result is shown in FIG. 3.

The content of IFN-γ in the serums of the female infant mice in the CCFM1269 group is significantly higher than that in the normal group (p<0.05), the concentration of IFN-γ in the serums of the female infant mice in the normal group is 0.74 ng/ml, and the concentration of IFN-γ in the serums of the female infant mice in the CCFM1269 group is 0.92 ng/ml.

IgG2a/IgE in the serums of the female infant mice in the CCFM1269 group is significantly higher than that in the normal group (p<0.05), the ratio of IgG2a to IgE in the serums of the female infant mice in the normal group is 0.60, and the ratio of IgG2a to IgE in the serums of the female infant mice in the CCFM1269 group is 0.81.

### Example 6: An Influence of B. longum subsp. infantis CCFM1269 on Expression Levels of T-Bet in Colons of Female Infant Mice

Grouping and molding of the mice are as same as those in example 3. After experiments, blood was collected and the mice were killed. Colons of the mice were taken and RNAs were extracted and were subjected to reverse transcription with a kit. The expression levels of T-bet in the colons of the mice in each group were determined. A detection result is shown in FIG. 4.

CCFM1269 can significantly improve the relative expression quantity of T-bet in the colons of the female infant mice (p<0.01), and the relative expression quantity in the CCFM1269 group is 1.45 times as much as that in the normal group.

### Example 7: An Influence of B. longum subsp. infantis CCFM1269 on Contents of Lymphocytes in Mesenteric Lymph Nodes of Male Infant Mice

After 8 6-week old male specific pathogen-free (SPF) BALB/C female mice and 4 6-week old SPF BALB/C male mice were fed 1 week under the condition of freely eating and drinking in a 12 h-12 h day/night cycle at a feeding room temperature of 22-24° C. and humidity of 40-60%, the female and male mice were mated in the proportion of 2:1; after the female mice were pregnant, the male mice were taken out; the gestation period was 3 weeks; and 1-week old infant mice after birth were subjected to intragastric administration, and were divided into a Control (intragastric administration with normal saline) and a CCFM1269 group (intragastric administration with the B. longum subsp. infantis CCFM1269 bacterial suspension).

Experiments were conducted after 1 week of adaptive feeding of animals, and the experiments lasted for 8 weeks. Specific treatments were as follows:

The Control: 1 week after birth, the mice were subjected to intragastric administration with 200 μL of normal saline as control; and

the B. longum subsp. infantis CCFM1269 group: 1 week after birth, the mice were subjected to intragastric administration with 10 μL of bacterial suspension, where the intragastric administration dose was 1×10⁹ CFU/mouse/day.

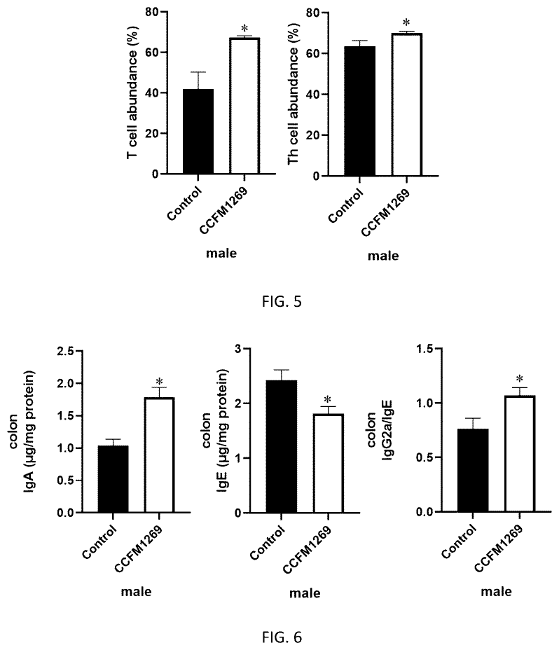

The mice were subjected to intragastric administration till they were 3-week old. After experiments, mesenteric lymph nodes of the male infant mice were taken and placed in a D-PBS to prepare a single-cell suspension. Through flow cytometry, T cells and B cells in the mesenteric lymph nodes were determined, and determination results were respectively shown in FIG. 5.

The percentage content of the T cells in the mesenteric lymph nodes of the male infant mice in the CCFM1269 group is significantly higher than that in the normal group (p<0.05), the content of the T cells in the mesenteric lymph nodes of the male infant mice in the normal group is 41.9%, and the content of the T cells in the mesenteric lymph nodes of the male infant mice in the CCFM1269 group is 67.3%.

The percentage content of the Th cells in the mesenteric lymph nodes of the male infant mice in the CCFM1269 group is significantly higher than that in the normal group (p<0.05), the content of the Th cells in the mesenteric lymph nodes of the male infant mice in the normal group is 63.24%, and the content of the Th cells in the mesenteric lymph nodes of the male infant mice in the CCFM1269 group is 70.11%.

### Example 8: An Influence of B. longum subsp. infantis CCFM1269 on Th1/Th2 Balance and a Content of IgA in Colons Of Male Infant Mice

Grouping of the mice is as same as that in example 3. After experiments, colons of the mice were taken and placed in phosphate-buffered saline and were homogenized. Homogenates of the colons of the mice in each group were determined through an ELISA kit. A detection result is shown in FIG. 6.

As shown in FIG. 6, the content of IgA in the colons of the male infant mice is significantly improved in the CCFM1269 group (p<0.01), the content of IgA in the homogenates of the colons in the normal group is 1.03 μg/mg protein, and the content of IgA in the homogenates of the colons in the CCFM1269 group is 1.78 μg/mg protein.

CCFM1269 significantly reduces the content of IgE in the colons of the male infant mice (p<0.05), the content of IgE in the homogenates of the colons in the normal group is 2.43 μg/mg protein, and the content of IgE in the homogenates of the colons in the CCFM1269 group is 1.81 μg/mg protein.

CCFM1269 significantly improves IgG2a/IgE of the male infant mice (p<0.05), the ratio of IgG2a to IgE in the homogenates of the colons in the normal group is 0.76, and the ratio of IgG2a to IgE in the homogenates of the colons in the CCFM1269 group is 1.07.

### Example 9: An Influence of B. longum subsp. infantis CCFM1269 on Th1/Th2 Balance in Serums of Male Infant Mice

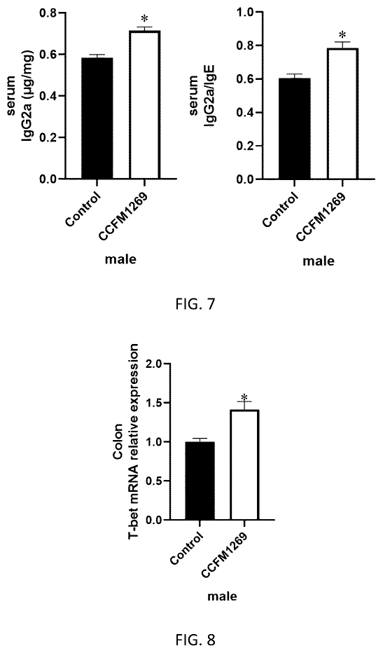

Grouping and treatment of the mice are as same as those in example 3. After experiments, blood was collected and the mice were killed. Th1/Th2-related cell factors in the serums of the mice in each group were determined through an ELISA kit. A detection result is shown in FIG. 7.

The level of IgG2a in the serums of the male infant mice in the CCFM1269 group is significantly improved (p<0.05), the concentration of IgG2a in the serums of the male infant mice in the normal group is 0.58 ng/ml, and the concentration of IgG2a in the serums of the male infant mice in the CCFM1269 group is 0.72 ng/ml.

IgG2a/IgE in the serums of the male infant mice in the CCFM1269 group is significantly improved (p<0.05), the ratio of IgG2a to IgE in the serums of the male infant mice in the normal group is 0.60, the ratio of IgG2a to IgE in the serums of the male infant mice in the CCFM1269 group is 0.79.

### Example 10: An Influence of B. longum subsp. infantis CCFM1269 On Expression Levels of T-Bet in Colons of Male Infant Mice

Grouping and molding of the mice are as same as those in example 3. After experiments, blood was collected and the mice were killed. Colons of the mice were taken and RNAs were extracted and were subjected to reverse transcription with a kit. The expression levels of T-bet in the colons of the mice in each group were determined. A detection result is shown in FIG. 8.

The transcription factor T-bet is a key factor that specifically regulates Th0 differentiation and plays a role of a Th1/Th2 transfer switch. T-bet transcription only emerges in a Th1 cell line. Therefore, it is believed that T-bet is selectively expressed in Th1 cells. T-bet, as a Th1 specific transcription factor, is selectively expressed in the Th1 cells, plays an important role in the development of the Th1 cells by initiating a Th1 genetic program, and inhibits synthesis of Th2 cell factors.

It can be known from FIG. 8 that CCFM1269 can significantly improve the relative expression quantity of T-bet in the colons of the male infant mice (p<0.05), and the relative expression quantity in the CCFM1269 group is 1.41 times as much as that in the normal group.

### Example 11: An Influence of B. longum subsp. infantis CCFM1269 on IgA-Coated Bacteria in Feces of Male Infant Mice

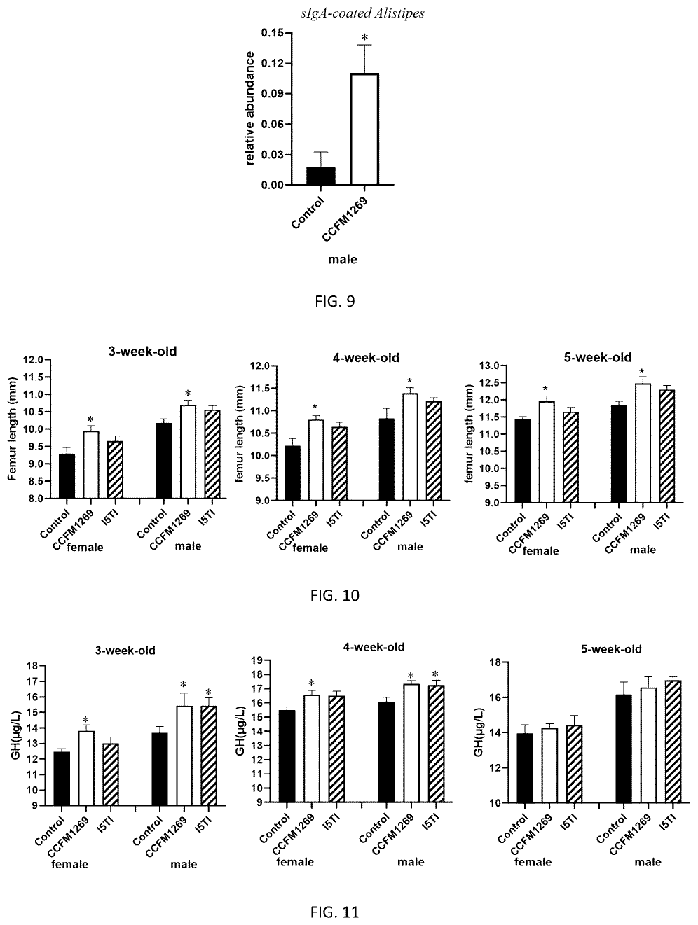

Grouping and molding of the mice are as same as those in example 3. After experiments, feces of the mice were collected, IgA-coated bacteria were enriched, genomic DNA in the feces was extracted through a FastDNA Spin Kit (MP Biomedicals in US), V3-V4 regions of the genomic DNA extracted were subjected to specific PCR amplification and 16S rDNA sequencing, and flora variations of the feces were analyzed. An analysis result is shown in FIG. 9.

It can be known from FIG. 9 that the relative abundance of the IgA-coated Alistipes in the Control of the male infant mice is 0.018, and the relative abundance of the IgA-coated Alistipes in the CCFM1269 group of the male infant mice is 0.11, so that the relative abundance of the IgA-coated Alistipes is significantly improved.

### Example 12: An Influence of B. longum subsp. infantis CCFM1269 on Femur Lengths of Female and Male Infant Mice

After 36 6-week old male specific pathogen-free (SPF) BALB/C female mice and 18 6-week old SPF BALB/C male mice were fed 1 week under the condition of freely eating and drinking in a 12 h-12 h day/night cycle at a feeding room temperature of 22-24° C. and humidity of 40-60%, the female and male mice were mated in the proportion of 2:1; after the female mice were pregnant, the male mice were taken out; the gestation period was 3 weeks; and 1-week old infant mice after birth were subjected to intragastric administration, and were divided into a Control (intragastric administration with normal saline), a CCFM1269 group, and an 15TI group (intragastric administration with the B. longum subsp. infantis 15TI, with the original number: FJSWXI5TIM1, which has been disclosed in a paper Research on Functional Genomes of B. longum subsp. infantis and Influence thereof on DSS Induced Colonitis). The mice were subjected to intragastric administration to 3-, 4-, and 5-week old.

Experiments were conducted after 1 week of adaptive feeding of animals, and the experiments lasted for 10 weeks. Specific treatments were as follows:

The Control: 1 week after birth, the mice were subjected to intragastric administration with 10 μl of normal saline; and

the B. longum subsp. infantis CCFM1269 group: 1 week after birth, the mice were subjected to intragastric administration with 10 μL of corresponding bacterial suspension, where the total intragastric administration dose was 1×10⁹ CFU/mouse/day.

The B. longum subsp. infantis 15TI group: 1 week after birth, the mice were subjected to intragastric administration with 10 μL of corresponding bacterial suspension, where the total intragastric administration dose was 1×10⁹ CFU/mouse/day.

With the change of week of the mice, the intragastric administration volume is increased according to the weight, but the total intragastric administration dose is kept at 1×10⁹ CFU/mouse/day and the mice were subjected to intragastric administration to 3-, 4-, and 5-week old. After the experiments, the whole blood of the female and male infant mice was collected and centrifuged to take serums. Femur lengths were measured with a vernier caliper. Determination results were respectively shown in FIG. 10.

It can be known from FIG. 10 that the femur lengths of the 3-week old female mice in the normal group are 9.30 mm, the femur lengths of the 3-week old female mice in the CCFM1269 group are 9.95 mm, and femur lengths of the 3-week old female mice in the 15TI group are 9.65 mm; the femur lengths of the 3-week old male mice in the normal group are 10.18 mm, the femur lengths of the 3-week old male mice in the CCFM1269 group are 10.70 mm, and femur lengths of the 3-week old male mice in the 15TI group are 10.56 mm; the femur lengths of the 4-week old female mice in the normal group are 10.24 mm, the femur lengths of the 4-week old female mice in the CCFM1269 group are 10.80 mm, and femur lengths of the 4-week old female mice in the 15TI group are 10.64 mm; the femur lengths of the 4-week old male mice in the normal group are 10.76 mm, the femur lengths of the 4-week old male mice in the CCFM1269 group are 11.39 mm, and femur lengths of the 4-week old male mice in the 15TI group are 11.21 mm; the femur lengths of the 5-week old female mice in the normal group are 11.28 mm, the femur lengths of the 5-week old female mice in the CCFM1269 group are 11.96 mm, and femur lengths of the 5-week old female mice in the 15TI group are 11.77 mm; and the femur lengths of the 5-week old male mice in the normal group are 11.84 mm, the femur lengths of the 5-week old male mice in the CCFM1269 group are 12.48 mm, and femur lengths of the 5-week old male mice in the 15TI group are 12.30 mm.

### Example 13: An Influence of B. longum subsp. infantis CCFM1269 on Levels of a Growth Hormone in Serums of Female and Male Infant Mice

Serums of the female and male mice were collected, and levels of the growth hormone in the serums of the mice in each group were determined through an ELISA kit. Determination results were respectively shown in FIG. 11.

The growth hormone can prompt growth, division growth of bones, cartilages, muscles and other tissue cells, and synthesis of proteins, so as to accelerate the growth and development of skeletons and muscles. The levels of the growth hormone in the serums of the 3-week and 4-week old female and male mice subjected to intragastric administration with CCFM1269 are significantly higher than those in the normal group; the concentration of the growth hormone in serums of the 3-week old female mice in the normal group is 12.48 μg/L, the concentration of the growth hormone in the serums of the 3-week old female mice in the CCFM1269 group is 13.82 μg/L, and the concentration of the growth hormone in the serums of the 3-week old female mice in the 15TI group is 13.00 μg/L; the concentration of the growth hormone in the serums of the 3-week old male mice in the normal group is 13.68 μg/L, the concentration of the growth hormone in the serums of the 3-week old male mice in the CCFM1269 group is 15.42 μg/L, and the concentration of the growth hormone in the serums of the 3-week old male mice in the 15TI group is 15.42 μg/L; the concentration of the growth hormone in the serums of the 4-week old female mice in the normal group is 15.49 μg/L, the concentration of the growth hormone in the serums of the 4-week old female mice in the CCFM1269 group is 16.58 μg/L, and the concentration of the growth hormone in the serums of the 4-week old female mice in the 15TI group is 16.35 μg/L; the concentration of the growth hormone in the serums of the 4-week male old mice in the normal group is 16.09 μg/L, the concentration of the growth hormone in the serums of the 4-week old male mice in the CCFM1269 group is 17.34 μg/L, and the concentration of the growth hormone in the serums of the 4-week old male mice in the 15TI group is 17.27 μg/L; and the difference among the 5-week old mice is insignificant.

### Example 14: An Influence of B. longum subsp. infantis CCFM1269 on Levels of an Insulin-Like Growth Factor 1 in Serums of Female and Male Infant Mice

Grouping and molding of the mice are as same as those in example 12. After experiments, blood was collected and the mice were killed. Levels of the insulin-like growth factor 1 in the serums of the mice in each group were determined through an ELISA kit. A detection result is shown in FIG. 12.

The insulin-like growth factor 1, also known as a “growth-promoting factor”, is significant to the growth of infants and the anabolic action continuously performed in adults, and prompts the anabolism of bones and keeps the normal structures and functions of the bones. The levels of the insulin-like growth factor 1 in the serums of the 4-week and 5-week old female and male mice subjected to intragastric administration with CCFM1269 are significantly higher than those in the normal group; the difference among the 3-week old mice is insignificant; the concentration of the insulin-like growth factor 1 in the serums of the 4-week old female mice in the normal group is 18.64 μg/L, the concentration of the insulin-like growth factor 1 in the serums of the 4-week old female mice in the CCFM1269 group is 22.34 μg/L, and the concentration of the insulin-like growth factor 1 in the serums of the 4-week old female mice in the 15TI group is 20.89 μg/L; and the concentration of the insulin-like growth factor 1 in the serums of the 4-week old male mice in the normal group is 19.77 μg/L, the concentration of the insulin-like growth factor 1 in the serums of the 4-week old male mice in the CCFM1269group is 23.03 μg/L, and the concentration of the insulin-like growth factor 1 in the serums of the 4-week old male mice in the 15TI group is 21.33 μg/L. The concentration of the insulin-like growth factor 1 in the serums of the 5-week female mice in the normal group is 25.41 μg/L, the concentration of the insulin-like growth factor 1 in the serums of the 5-week female mice in the CCFM1269 group is 28.58 μg/L, and the concentration of the insulin-like growth factor 1 in the serums of the 5-week female mice in the 15TI group is 28.39 μg/L; and the concentration of the insulin-like growth factor 1 in the serums of the 5-week male mice in the normal group is 26.93 μg/L, the concentration of the insulin-like growth factor 1 in the serums of the 5-week male mice in the CCFM1269 group is 30.92 μg/L, and the concentration of the insulin-like growth factor 1 in the serums of the 5-week male mice in the 15TI group is 29.26 μg/L.

### Example 15: An Influence of B. longum subsp. infantis CCFM1269 on Levels of Osteoprotegerin in Serums of Female and Male Infant Mice

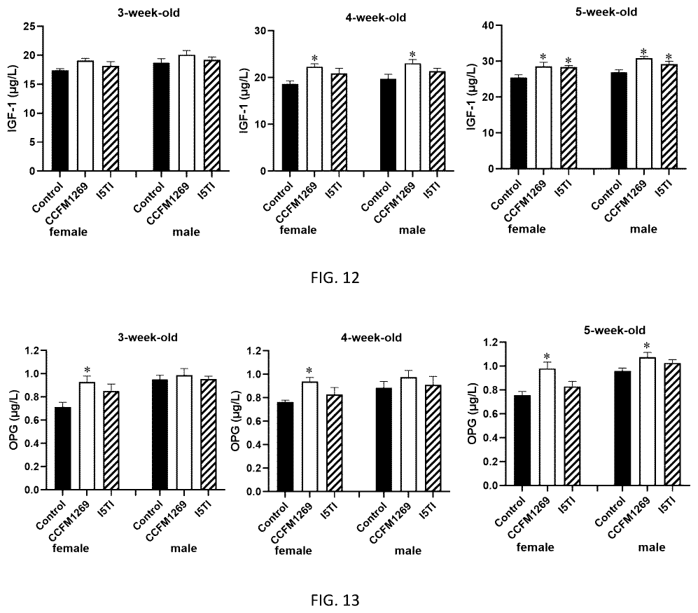

Grouping and molding of the mice are as same as those in example 12. After experiments, blood was collected and the mice were killed. Levels of osteoprotegerin in the serums of the mice in each group were determined through an ELISA kit. A detection result is shown in FIG. 13.

The osteoprotegerin can inhibit the occurrence of osteoclasts and prompt apoptosis of mature osteoclasts. The levels of the osteoprotegerin in the serums of the 3-, 4-, and 5-week old female and male mice subjected to intragastric administration with CCFM1269are significantly higher than those in the normal group; the concentration of the osteoprotegerin in the serums of the 3-week old female mice in the normal group is 0.71 μg/L, the concentration of the osteoprotegerin in the serums of the 3-week old female mice in the CCFM1269 group is 0.93 μg/L, and the concentration of the osteoprotegerin in the serums of the 3-week old female mice in the 15TI group is 0.85 μg/L; the concentration of the osteoprotegerin in the serums of the 3-week old male mice in the normal group is 0.95 μg/L, the concentration of the osteoprotegerin in the serums of the 3-week old male mice in the CCFM1269 group is 0.98 μg/L, and the concentration of the osteoprotegerin in the serums of the 3-week old male mice in the 15TI group is 0.95 μg/L; the concentration of the osteoprotegerin in the serums of the 4-week old female mice in the normal group is 0.76 μg/L, the concentration of the osteoprotegerin in the serums of the 4-week old female mice in the CCFM1269 group is 0.94 μg/L, and the concentration of the osteoprotegerin in the serums of the 4-week old female mice in the 15TI group is 0.83 μg/L; the concentration of the osteoprotegerin in the serums of the 4-week male old mice in the normal group is 0.88 μg/L, the concentration of the osteoprotegerin in the serums of the 4-week old male mice in the CCFM1269 group is 0.98 μg/L, and the concentration of the osteoprotegerin in the serums of the 4-week old male mice in the 15TI group is 0.91 μg/L; the concentration of the osteoprotegerin in the serums of the 5-week old female mice in the normal group is 0.76 μg/L, the concentration of the osteoprotegerin in the serums of the 5-week old female mice in the CCFM1269 group is 0.98 μg/L, and the concentration of the osteoprotegerin in the serums of the 5-week old female mice in the 15TI group is 0.83 μg/L; and the concentration of the osteoprotegerin in the serums of the 5-week old male mice in the normal group is 0.96 μg/L, the concentration of the osteoprotegerin in the serums of the 5-week old male mice in the CCFM1269 group is 1.08 μg/L, and the concentration of the osteoprotegerin in the serums of the 5-week old male mice in the 15TI group is 1.02 μg/L.

### Example 16: An Influence of B. longum subsp. infantis CCFM1269 on Levels of Osteocalcin in Serums of Female and Male Infant Mice

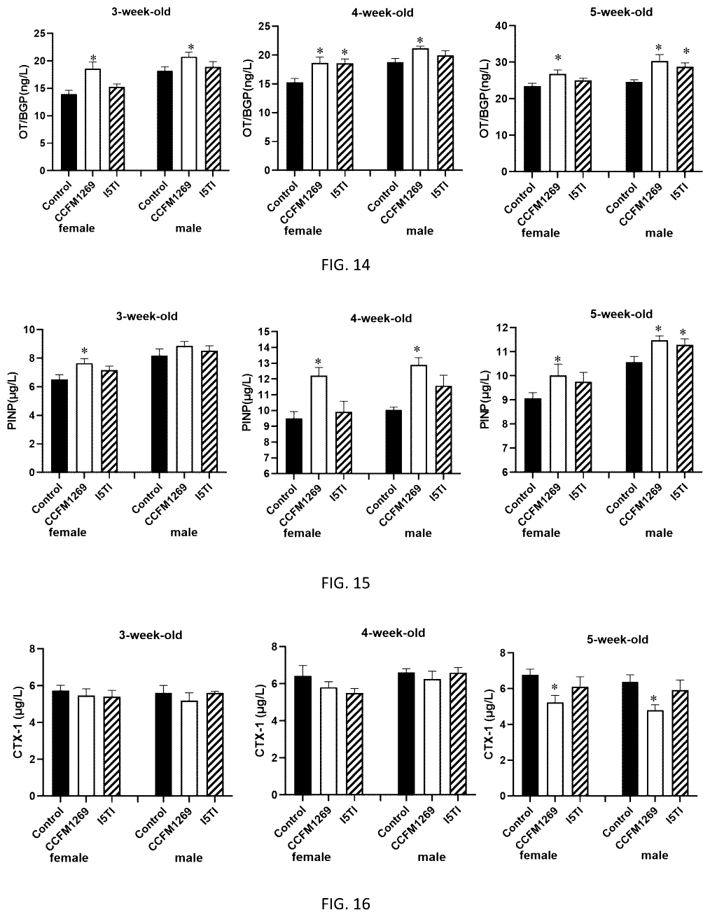

Grouping and molding of the mice are as same as those in example 12. After experiments, blood was collected and the mice were killed. Levels of osteocalcin in the serums of the mice in each group were determined through an ELISA kit. A detection result is shown in FIG. 14.

The osteocalcin is a sensitive mark which reflects the function of the osteoblasts. The levels of the osteocalcin in the serums of the 3-, 4-, and 5-week old female and male mice subjected to intragastric administration with CCFM1269 are significantly higher than those in the normal group; the concentration of the osteocalcin in the serums of the 3-week old female mice in the normal group is 13.94 μg/L, the concentration of the osteocalcin in the serums of the 3-week old female mice in the CCFM1269 group is 18.57 μg/L, and the concentration of the osteocalcin in the serums of the 3-week old female mice in the 15TI group is 15.24 μg/L; the concentration of the osteocalcin in the serums of the 3-week old male mice in the normal group is 18.21 μg/L, the concentration of the osteocalcin in the serums of the 3-week old male mice in the CCFM1269 group is 20.75 μg/L, and the concentration of the osteocalcin in the serums of the 3-week old male mice in the 15TI group is 18.92 μg/L; the concentration of the osteocalcin in the serums of the 4-week old female mice in the normal group is 15.24 μg/L, the concentration of the osteocalcin in the serums of the 4-week old female mice in the CCFM1269 group is 18.65μg/L, and the concentration of the osteocalcin in the serums of the 4-week old female mice in the 15TI group is 18.56 μg/L; the concentration of the osteocalcin in the serums of the 4-week male old mice in the normal group is 18.75 μg/L, the concentration of the osteocalcin in the serums of the 4-week old male mice in the CCFM1269 group is 21.16 μg/L, and the concentration of the osteocalcin in the serums of the 4-week old male mice in the 15TI group is 19.95 μg/L; the concentration of the osteocalcin in the serums of the 5-week old female mice in the normal group is 23.49 μg/L, the concentration of the osteocalcin in the serums of the 5-week old female mice in the CCFM1269 group is 26.82 μg/L, and the concentration of the osteocalcin in the serums of the 5-week old female mice in the 15TI group is 25.02 μg/L; and the concentration of the osteocalcin in the serums of the 5-week old male mice in the normal group is 24.68 μg/L, the concentration of the osteocalcin in the serums of the 5-week old male mice in the CCFM1269group is 30.30 μg/L, and the concentration of the osteocalcin in the serums of the 5-week old male mice in the 15TI group is 28.75 μg/L.

### Example 17: An Influence of B. longum subsp. infantis CCFM1269 on Levels of N-Terminal Propeptides of Type I Collagens in Serums of Female and Male Infant Mice

Grouping and molding of the mice are as same as those in example 12. After experiments, blood was collected and the mice were killed. Levels of N-terminal propeptides of type I collagens in the serums of the mice in each group were determined through an ELISA kit. A detection result is shown in FIG. 15.

The amino-terminal peptide of the type I procollagen in the serum is a highly sensitive marker that reflects bone formation. The levels of the amino-terminal peptides of the type I procollagens in the serums of the 3-, 4-, and 5-week old female and male mice subjected to intragastric administration with CCFM1269 are significantly higher than those in the normal group; the concentration of the amino-terminal peptides of the type I procollagens in the serums of the 3-week old female mice in the normal group is 6.51 μg/L, the concentration of the amino-terminal peptides of the type I procollagens in the serums of the 3-week old female mice in the CCFM1269 group is 7.65 μg/L, and the concentration of the amino-terminal peptides of the type I procollagens in the serums of the 3-week old female mice in the 15TI group is 7.16 μg/L; the concentration of the amino-terminal peptides of the type I procollagens in the serums of the 3-week old male mice in the normal group is 8.19 μg/L, the concentration of the amino-terminal peptides of the type I procollagens in the serums of the 3-week old male mice in the CCFM1269 group is 8.86 μg/L, and the concentration of the amino-terminal peptides of the type I procollagens in the serums of the 3-week old male mice in the 15TI group is 8.53 μg/L; the concentration of the amino-terminal peptides of the type I procollagens in the serums of the 4-week old female mice in the normal group is 8.95 μg/L, the concentration of the amino-terminal peptides of the type I procollagens in the serums of the 4-week old female mice in the CCFM1269 group is 12.21 μg/L, and the concentration of the amino-terminal peptides of the type I procollagens in the serums of the 4-week old female mice in the 15TI group is 9.92 μg/L; the concentration of the amino-terminal peptides of the type I procollagens in the serums of the 4-week male old mice in the normal group is 10.04 μg/L, the concentration of the amino-terminal peptides of the type I procollagens in the serums of the 4-week old male mice in the CCFM1269 group is 12.90 μg/L, and the concentration of the amino-terminal peptides of the type I procollagens in the serums of the 4-week old male mice in the 15TI group is 11.57 μg/L; the concentration of the amino-terminal peptides of the type I procollagens in the serums of the 5-week old female mice in the normal group is 9.07 μg/L, the concentration of the amino-terminal peptides of the type I procollagens in the serums of the 5-week old female mice in the CCFM1269 group is 10.01 μg/L, and the concentration of the amino-terminal peptides of the type I procollagens in the serums of the 5-week old female mice in the 15TI group is 9.75 μg/L; and the concentration of the amino-terminal peptides of the type I procollagens in the serums of the 5-week old male mice in the normal group is 10.56 μg/L, the concentration of the amino-terminal peptides of the type I procollagens in the serums of the 5-week old male mice in the CCFM1269 group is 11.47 μg/L, and the concentration of the amino-terminal peptides of the type I procollagens in the serums of the 5-week old male mice in the 15TI group is 11.28 μg/L.

### Example 18: An Influence of B. longum subsp. infantis CCFM1269 on Levels of C-Terminal Peptides of Type I Collagens in Serums of Female and Male Infant Mice

Grouping and molding of the mice are as same as those in example 12. After experiments, blood was collected and the mice were killed. Levels of C-terminal peptides of type I collagens in the serums of the mice in each group were determined through an ELISA kit. A detection result is shown in FIG. 16.

The type I collagen crosslinked C-terminal peptide is the most sensitive and specific index which reflects bone absorption. There is no significant difference between the C-terminal peptides of type I collagens in the serums of the 3-and 4-week old female and male mice subjected to intragastric administration with CCFM1269 and those in the normal group. This index in the serums of the 5-week old female and male mice are significantly lower than those in the normal group, indicating that CCFM1269 can effectively reduce the bone absorption degree, which facilitates the skeletal development of the mice; the concentration of the C-terminal peptides of type I collagens in the serums of the 5-week old female mice in the normal group is 6.78 μg/L, the concentration of the C-terminal peptides of type I collagens in the serums of the 5-week old female mice in the CCFM1269 group is 5.23 μg/L, and the concentration of the C-terminal peptides of type I collagens in the serums of the 5-week old female mice in the 15TI group is 6.11 μg/L; and the concentration of the C-terminal peptides of type I collagens in the serums of the 5-week old male mice in the normal group is 6.38 μg/L, the concentration of the C-terminal peptides of type I collagens in the serums of the 5-week old male mice in the CCFM1269 group is 4.79 μg/L, and the concentration of the C-terminal peptides of type I collagens in the serums of the 5-week old male mice in the 15TI group is 5.91 μg/L.

Although disclosed with the preferred examples above, the disclosure is not limited by the examples. Any person skilled in the art may make various alternations and modifications without departing the spirit and scope of the disclosure. Therefore, the scope of protection of the disclosure should be subject to the definition in the claims.

What is claimed is:

1. A composition, comprising Bifidobacterium longum subsp. infantis CCFM1269 or a culture thereof, wherein the B. longum subsp. infantis CCFM1269 has been preserved in Guangdong Microbial Culture Collection Center (GDMCC) on Sep. 26, 2022, with a preservation number of GDMCC No: 62839 and a preservation address: Floor 5, Building 59, Yard 100, Middle Xianlie Road, Guangzhou; and the composition is a probiotic preparation, a leavening agent, a health care product, or a medicine.

2. The probiotic preparation according to claim 1, wherein a viable count of the B. longum subsp. infantis CCFM1269 in the probiotic preparation is not lower than 1×10⁹ CFU/ml or 1×10⁹ CFU/g.

3. The composition according to claim 1, wherein an existing form of the B. longum subsp. infantis CCFM1269 comprises a bacteria solution, a freeze-dried powder, or a fermented composition.

4. The composition according to claim 1, wherein objects using the composition are mammals in an early life stage; and the composition is used to improve the levels of Th1/Th2 related cell factors and immune globulins in colons of the mammals in the early life stage, improve the bone density, enhance the skeletal development, and regulate the intestinal flora.

5. A method for enhancing immunity, improving bone density in an auxiliary manner, enhancing skeletal development, and adjusting the intestinal flora, comprising ingesting the composition according to claim 1 into gastrointestinal environments of individuals.

6. The method according to claim 5, wherein the enhancing immunity refers to regulating Th1/Th2 balance and IgA synthesis.

7. The method according to claim 5, wherein the individuals comprise mammals in the early life stage; and the enhancing the immunity comprises improving the levels of Th1/Th2 related cell factors and immune globulins in the colons.

8. The method according to claim 6, wherein the regulating the Th1/Th2 balance and IgA synthesis comprises at least one of the following effects:
(1) improving the percentage contents of B cells, T cells, and Th cells in mesenteric lymph nodes of mammals in the early life stage;
(2) improving the contents of IFN-γ, IgG2a, IgA, and sIgA in the colons of mammals in the early life stage and the ratio of IgG2a/IgE;
(3) improving the content of IFN-γ in serums of mammals in the early life stage and the ratio of IgG2a/IgE;
(4) reducing the contents of IL-4 and IgE in the colons of male mammals in the early life stage;
(5) improving the expression level of T-bet mRNA in the colons of mammals in the early life stage; and
(6) improving the relative abundance of IgA-coated Alistipes in feces of male mammals in the early life stage.

9. The method according to claim 6, wherein the enhancing the skeletal development refers to improving the femur length and the skeletal steady stage in the early life stage, comprising at least one of the following effects:
(1) improving the femur lengths of mammals in the early life stage;
(2) improving the levels of a growth hormone in the serums of mammals in the early life stage;
(3) improving the levels of an insulin-like growth factor 1 in the serums of mammals in the early life stage;
(4) improving the levels of osteoprotegerin in the serums of mammals in the early life stage;
(5) improving the levels of osteocalcin in the serums of mammals in the early life stage;
(6) improving the levels of N-terminal propeptides of type I collagens in the serums of mammals in the early life stage; and
(7) reducing the levels of C-terminal peptides of the type I collagens in the serums of mammals in the early life stage.

10. The method according to claim 5, wherein a viable count of the B. longum subsp. infantis CCFM1269 in the composition is not lower than 1×10⁹ CFU/g.

11. The application according to claim 10, wherein a form of the B. longum subsp. infantis CCFM1269 in the composition comprises a bacteria solution, a freeze-dried powder, or a fermented composition.
